(19) United States
(12) Patent Application Publication
(10) Pub. No.: US 2026/0017791 A1
(43) Pub. Date: Jan. 15, 2026

(54) ENDOSCOPIC SYSTEMS AND METHODS TO RE-IDENTIFY POLYPS USING MULTIVIEW INPUT IMAGES

(71) Applicant: Verily Life Sciences LLC, Dallas, TX (US)

(72) Inventors: Yotam Intrator, Beer Yakov (IL); Natalia Aizenberg, Netanya (IL); Israel Or Weinstein, Tel Aviv (IL); Roman Goldenberg, Haifa (IL)

(21) Appl. No.: 19/137,486

(22) PCT Filed: Dec. 28, 2023
(86) PCT No.: PCT/US2023/086295

§ 371 Date: Jun. 10, 2025

Related U.S. Application Data
(60) Provisional application No. 63/482,421, filed on Jan. 31, 2023.
(60) Provisional application No. 63/487,729, filed on Mar. 1, 2023.

(51) Int. Cl. G06T 7/00; A61B 1/00; A61B 1/005; A61B 1/31

(52) U.S. Cl. CPC G06T 7/0014; A61B 1/000096; A61B 1/005; A61B 1/31; G06T 2207/10068; G06T 2207/20081; G06T 2207/30028; G06T 2207/30096

(57) ABSTRACT

An endoscopic system, methods, and a machine-learned model trained to represent an area of interest in a body, such as a polyp, are described. In an embodiment, the machine-learned model uses data comprising multiple images of the area of interest as a vector in a latent space. In an embodiment, the methods include comparing a first plurality of images of a portion of a body and a second plurality of images of a portion of the body to determine a likelihood that the first portion is the second portion.

### CROSS-REFERENCE TO RELATED APPLICATIONS

This application claims the benefit of U.S. Provisional Application 63/482,421, filed Jan. 31, 2023, and U.S. Provisional Application 63/487,729, filed Mar. 1, 2023, the contents both of which are incorporated by reference.

### TECHNICAL FIELD

This disclosure relates generally to endoscopic systems and methods for analyzing a portion of a body and, in certain examples, endoscopic systems and methods for re-identifying portions of a body.

### BACKGROUND INFORMATION

During colonoscopy procedures, it is common to lose visibility of a detected polyp, specifically during tool insertion for polyp management. In such cases, the endoscopist can often spend precious clinical time finding and re-identifying the “lost” polyp. Low confidence in polyp re-identification leads to inefficiency due to searching for a polyp already found. False re-identification may result in missing a polyp the endoscopist would have otherwise chosen to manage. The same clinical situation may occur when deciding to manage a polyp detected during the insertion phase versus waiting until withdrawal.

### BRIEF DESCRIPTION OF THE DRAWINGS

Non-limiting and non-exhaustive embodiments of the claimed subject matter are described with reference to the following figures, wherein like reference numerals refer to like parts throughout the various views unless otherwise specified. Not all instances of an element are necessarily labeled so as not to clutter the drawings where appropriate. The drawings are not necessarily to scale, emphasis instead being placed upon illustrating the principles being described.

FIG. 1A illustrates a colonoscopy tower system, in accordance with an embodiment of the disclosure.

FIG. 1B illustrates an endoscopy video assistant capable of generating a colonoscopy user-interface for identification and re-identification of an area of interest in a body, in accordance with an embodiment of the disclosure.

FIG. 2 is a schematic illustration of a method according to an embodiment of the present disclosure.

FIG. 3 is a block diagram of a method according to an embodiment of the present disclosure.

FIG. 4 is a comparison of TPR with FPR for early fusion methods, according to embodiments of the present disclosure, and late fusion.

FIG. 5 is a functional block diagram illustrating a demonstrative computing device for implementing an endoscopy video assistant, in accordance with any embodiment of the disclosure.

### DETAILED DESCRIPTION

Embodiments of endoscopic systems and methods for analyzing a portion of a body are described. In the following description numerous specific details are set forth to provide a thorough understanding of the embodiments. One skilled in the relevant art will recognize, however, that the techniques described herein can be practiced without one or more of the specific details, or with other methods, components, materials, etc. In other instances, well-known structures, materials, or operations are not shown or described in detail to avoid obscuring certain aspects.

Reference throughout this specification to “one embodiment” or “an embodiment” means that a particular feature, structure, or characteristic described in connection with the embodiment is included in at least one embodiment of the present invention. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

Some portions of the detailed description that follow are presented in terms of algorithms and symbolic representations of operations on data bits within a computer memory. These algorithmic descriptions and representations are the means used by those skilled in the data processing arts to most effectively convey the substance of their work to others skilled in the art. An algorithm is here, and generally, conceived to be a self-consistent sequence of steps leading to a desired result. The steps are those requiring physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise as apparent from the following discussion, it is appreciated that throughout the description, discussions utilizing terms such as “selecting”, “identifying”, “capturing”, “adjusting”, “analyzing”, “determining”, “estimating”, “generating”, “comparing”, “modifying”, “receiving”, “providing”, “displaying”, “interpolating”, “outputting”, or the like, refer to the actions and processes of a computer system, or similar electronic computing device, that manipulates and transforms data represented as physical (e.g., electronic) quantities within the computer system's registers and memories into other data similarly represented as physical quantities within the computer system memories or registers or other such as information storage, transmission, or display devices.

The algorithms and displays presented herein are not inherently related to any particular computer or other apparatus. Various general-purpose systems can be used with programs in accordance with the teachings herein, or it can prove convenient to construct a more specialized apparatus to perform the required method steps. The required structure for a variety of these systems will appear from the description below. In addition, embodiments of the present disclosure are not described with reference to any particular programming language. It will be appreciated that a variety of programming languages can be used to implement the teachings of the disclosure as described herein.

Described herein are embodiments of an apparatus, a system, and a method for re-identifying an area of interest within or on a body based on image or video data. Specifically, the re-identification of a portion of a body will be discussed in the context of a colonoscopy. However, it is appreciated that re-identification of an area of interest is not limited to colonoscopies and that re-identification with other types of endoscopy procedures may also be determined with the embodiments described herein. Accordingly, it is appreciated specific instances of the terms “colonoscopy”, “colonoscope”, or “colon” may be swapped throughout the detailed description for their more generic counterparts of “endoscopy,” “endoscope,” or “cavity or tubular anatomical structure,” respectively. It is further appreciated that endoscopy procedures are not limited to examination and/or exploration of the colon, but rather is generally applicable to endoscopy procedures in which the gastrointestinal tract (such as in identifying ulcers), the respiratory tract, the ear, the urinary tract, the female reproductive system, cavities that are normally closed but have been opened via an incision, or other hollow organs or cavities of a body are explored, examined, and/or have one or more surgical procedures performed therein or thereon with the aid of a medical instrument including a camera that can be introduced into the body.

Endoscopy procedures typically include multiple phases or stages defined by actions (or inaction) performed with an endoscope. The phases may be characterized by movement of the endoscope (or lack thereof), elapsed duration of a given phase, medical procedure performed with the endoscope (e.g., cutting, resection, cauterization, or the like), or any other distinctive operation performed during the endoscopy procedure. For example, a colonoscopy may include a forward phase, a stagnant phase, a backward phase, and a resection phase. During the forward phase a colonoscope is predominantly moved forward (i.e., +z direction) through the rectum until reaching the end of the colon, known as the cecum. During the forward phase, polyps may be identified. Once the cecum has been examined, the backward phase of the colonoscopy occurs in which the colonoscope is slowly extracted (i.e., −z direction) from the body.

During the backward phase, the goal is typically to detect, examine, and remove polyps. A user may wish to remove all identified polyps, such as all polyps identified during the forward phase. However, re-identifying of all polyps identified in the forward phase may be difficult even for experienced endoscopists.

Described herein are embodiments that may be implemented in an apparatus, system, or method to re-identify polyps or other areas of interest, such as re-identify polyps during a backwards phase that were previously identified in a forward phase.

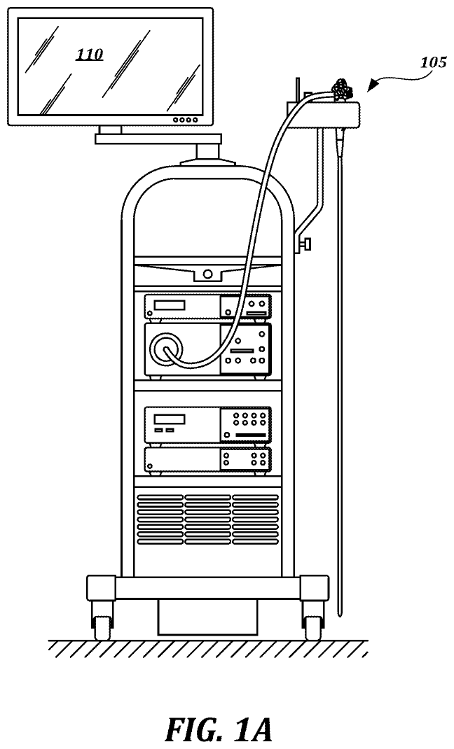

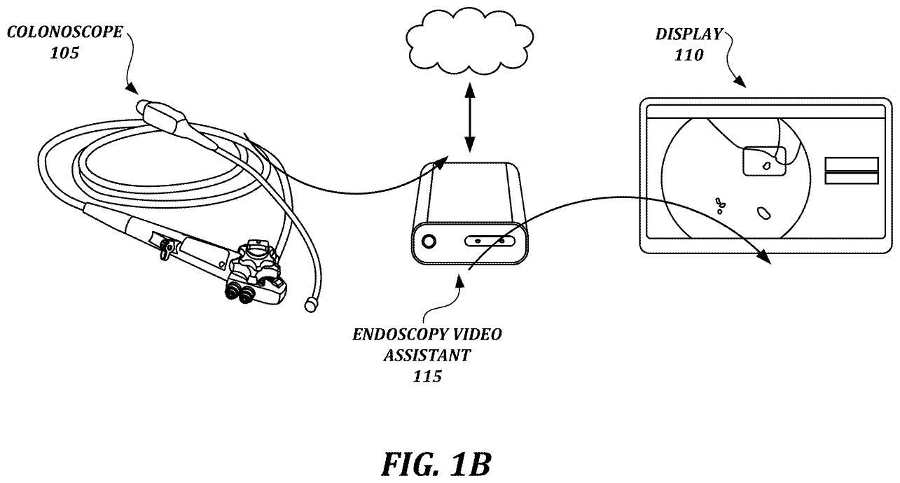

FIG. 1A illustrates an endoscopic system 100, in accordance with an embodiment of the disclosure. FIG. 1A illustrates an example endoscopic system in which re-identification of an area of interest of a body may be provided (e.g., in situ). System 100 includes an endoscope or colonoscope 105 coupled to a display 110 for capturing images of a colon and displaying a live video feed of the colonoscopy procedure. In an embodiment, the display 110 is optional. In one embodiment, the image or video analysis and user interface overlays described herein may be performed and generated by a processing box that plugs in between the colonoscope 105 and display 110.

FIG. 1B illustrates an example endoscopy video assistant (EVA) 115 capable of re-identifying a region of interest, and generating a colonoscopy user interface (UI) overlay described herein. EVA 115 may include the necessary processing hardware and software, including machine learning models, to perform the real-time image processing and UI overlays. For example, EVA 115 may include a data storage, a general-purpose processor, graphics processor, and video input/output (I/O) interfaces to receive a live video feed from colonoscope 105 and output the live video feed within a UI that overlays various visual aids and data associated with the colonoscopy or other endoscopy procedures. In some embodiments, EVA 115 may further include a network connection for offloading some of the image processing and/or reporting and saving data for individual patient recall and/or longitudinal, anonymized studies. The colonoscopy UI may include the live video feed reformatted, parsed, or scaled into a video region, or may be a UI overlay on top of an existing colonoscopy monitor feed to maintain the original format, resolution, and integrity of the colonoscopy live video feed.

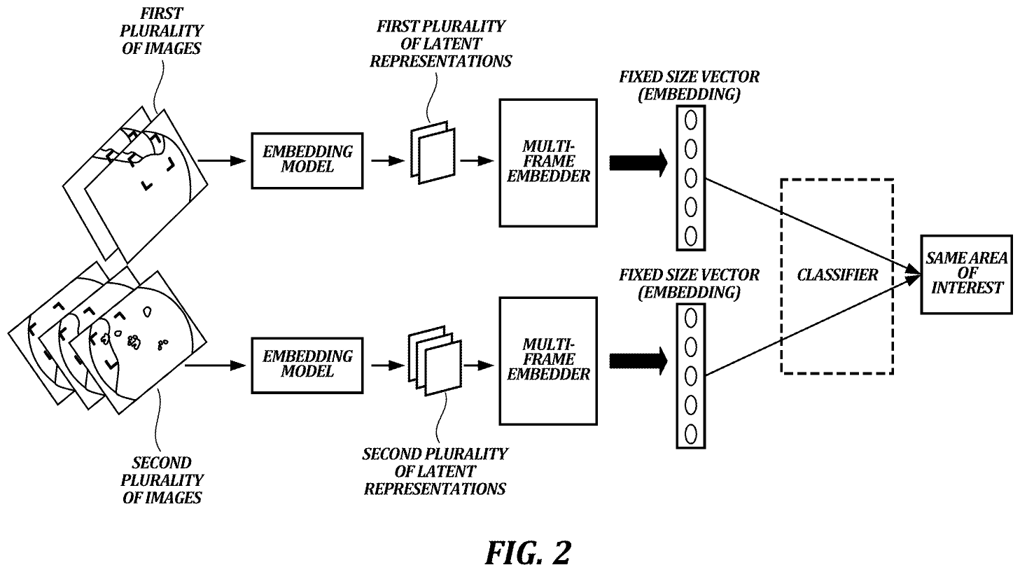

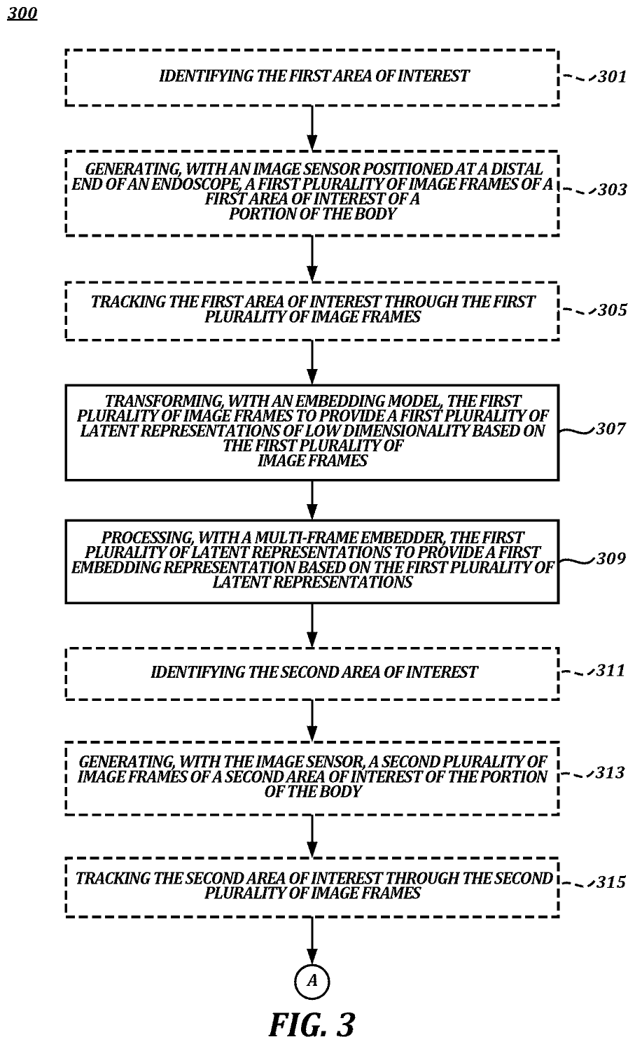

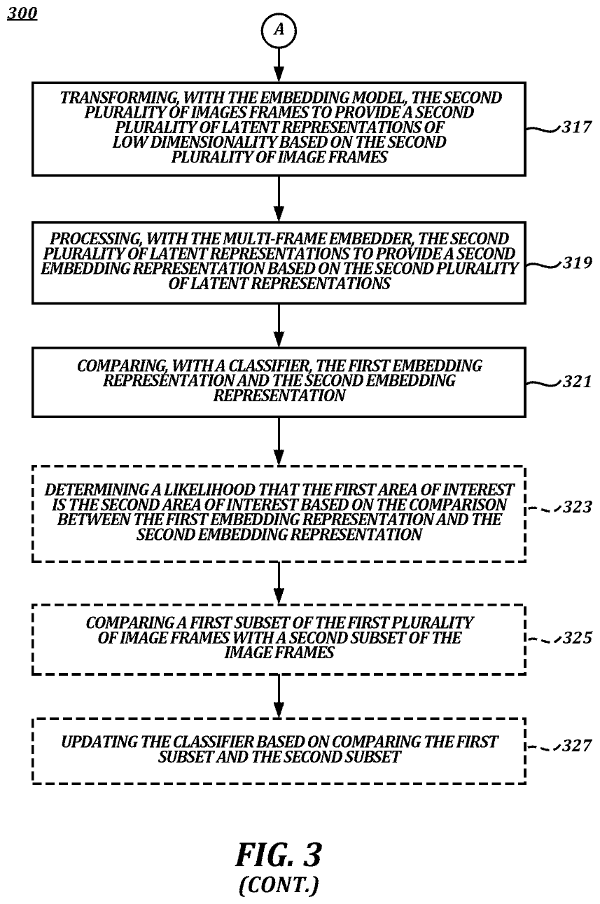

In an embodiment, the EVA 115 includes one or more models, modules, and the like for performing one or more of the methods of the present disclosure, such as one or more models, modules, and the like saved to memory. Accordingly, in an embodiment, the EVA 115 includes an embedding model, a multi-frame embedder, and a classifier, which are discussed further herein with respect to FIGS. 2 and 3.

In another aspect, the present disclosure provides a machine-learned model trained to re-identify an area of interest in a body, such as a polyp or ulcer, wherein the machine-learned model uses data comprising multiple images of the area of interest as a vector in a latent space. In this regard, the model leverages, for example, a video modality, which inherently provides multi-image input for each area of interest, e.g., a polyp. While polyps are discussed further herein as areas of interest, it will be understood that other types of areas of interest, such as ulcers, are possible and within the scope of the present disclosure.

In an embodiment, the learned representation contains information inferred from polyp images, and can be directly compared to vectors representing other polyps, via distance calculation.

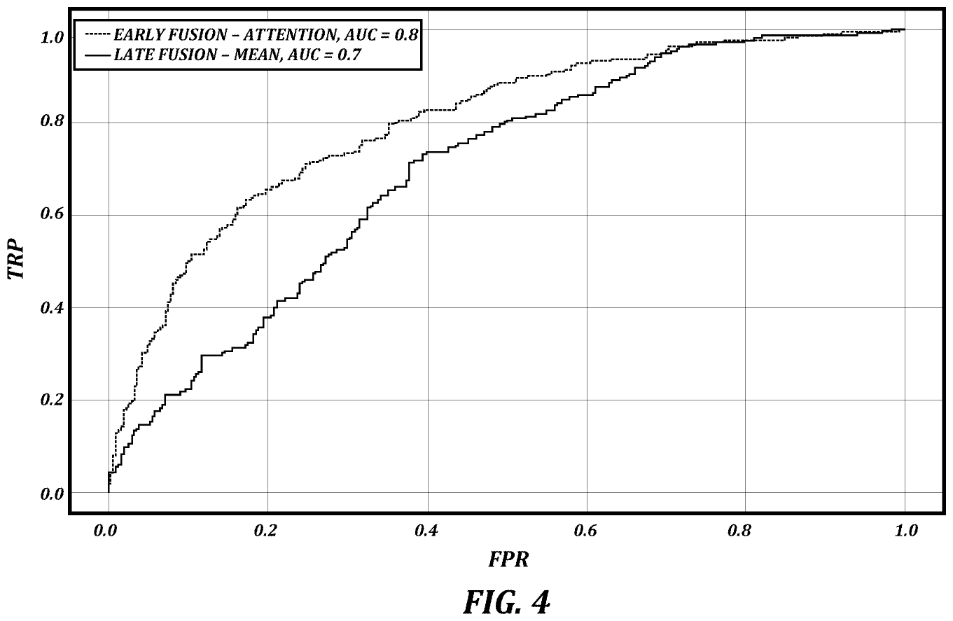

In an embodiment, the multi-image input is fused with transformer architecture (“early fusion”), to directly learn a single representation vector for multiple images, rather than applying “late fusion” heuristics on top of single image representations. See, e.g., FIG. 4.

Lack of labeled data for re-identification is addressed, in an embodiment, by using a self-supervised approach, referred to as contrastive learning, which allows using unlabeled data for optimization. In an embodiment, the self-supervised approach is SimCLR. With this approach, the systems and methods of the present disclosure use the same polyp video segments, for both positive and negative examples without any additional labeling.

In an embodiment, a first component is an embedding model, namely, a model that transforms an input image (frame) to a latent representation of low dimensionality. Given a video of a single polyp, this model may process each frame in the stream, and output a single embedding vector for each frame.

In an embodiment, a second component is a multi-frame embedder, which receives as input the embedding vectors of multiple frames, and mutually processes them to get a single embedding representing the polyp as a combination of all provided frames. In an embodiment, the processing of the vectors is done using an attention model (such as a transformer), which allows dynamic interpretation of the most distinctive features for any given input. In an embodiment, the output of the multi-frame embedder is a single vector for each polyp.

In an embodiment, a third component is a classifier, which takes as input two vectors of multi-frame representations and, based on the cosine similarity score, determines whether they belong to the same polyp or not.

The mutual processing of the features extracted from different frames and viewpoints allows a more robust and consistent performance, in comparison to the single frame approach.

For training these models, a polyp detection annotation dataset can be used. While this dataset may be quite large, it may also lack re-identification annotation. To utilize it for training, it may be assumed that all annotated polyps are unique and use self-learning via contrastive loss, which does not require hard negatives and can be robust to the false negative examples also used. However, instead of using augmentation for true positives (as is customary in Contrastive Learning), videos, or other multi-frame image sets are used to develop a sampling strategy for generating true positives from different views of the same area of interest, e.g., polyp. While a polyp detection annotation dataset (i.e., a data set including manually annotated polyps) is described, it will be understood that, in certain embodiments, automatically generated detections, which will allow for a fully automated process, without relying on any manual annotations are possible and within the scope of the present disclosure.

As an example, a model according to an embodiment of the present disclosure was trained on 11,240 polyp video segments.

Performance was evaluated on a smaller labeled set containing 444 polyp segment pairs (198 positive and 246 negative). Each pair was extracted from the same procedure, and labeled by an experienced endoscopist as either the same or two different polyps. Area under the curve (AUC) was calculated for a receiver operating characteristic (ROC) over the normalized vector distance for each of the pairs. An additional 77 pairs were annotated by two experienced endoscopists to evaluate interobserver disagreement rate.

The ReID model according to the present disclosure achieved 0.8 AUC ROC when tested on labeled polyp pairs.

Methods of the present disclosure will now be discussed with respect to FIGS. 2 and 3.

FIG. 2 is a schematic illustration of a method according to an embodiment of the present disclosure. As shown, the method includes use of a first plurality of image frames of a first area of interest, shown here highlighted with brackets around the first area of interest, and a second plurality of images of a second area of interest, also shown highlighted in brackets. As discussed further herein, in an embodiment, the first plurality of image frames include image frames from a video feed of a colonoscopy obtained during an insertion or forward phase of a procedure, whereas the second plurality of image frames include image frames of the video feed obtained during a retraction or backward phase of the procedure.

In the illustrated embodiment, the method includes use of an embedding model to provide a first plurality of latent representations based on the first plurality of image frames. As also shown, the method includes use of the embedding model to provide a second plurality of latent representations based on the second plurality of image frames. Such use of the embedding model can include transforming the first and second pluralities of images to provide latent representations having low dimensionality, which comprise salient information, features, or representations of the areas of interest.

As also shown, the method includes use of a multi-frame embedder to provide embedding representations based on the first and second pluralities of latent representations. In an embodiment, such use includes processing the latent representations with the multi-frame embedder. In an embodiment the embedding representations are fixed-size vectors, such as a single vector for a first embedding representation based on the first plurality of latent representations, and a single vector for a second embedding representation based on the second plurality of latent representations.

The illustrated method also shows a comparison between the first embedding representation and the second embedding representation. In an embodiment, the comparison includes use of a classifier. As discussed further herein, in an embodiment, such a comparison includes determining or generating a likelihood that the first area of interest is the second area of interest based on the comparison between the first embedding representation and the second embedding representation. In an embodiment, comparing, with the classifier, the first embedding representation and the second embedding representation comprises generating a cosine similarity score of the first embedding representation and the second embedding representation. As discussed further herein, such a cosine similarity score may be used to determine the likelihood that the first area of interest is the second area of interest.

FIG. 3 is a block diagram of a method 300 according to an embodiment of the present disclosure. In an embodiment, method 300 is configured to operate using and/or can be implemented using endoscopic system 100 discussed further herein with respect to FIGS. 1A and 1B or more generally with computing device discussed further herein with respect to FIG. 6. In an embodiment, method 300 is an example of the method illustrated and discussed further herein with respect to FIG. 2.

In the same or other embodiments, the method 300 may be a computer-implemented method including instructions provided by at least one machine-accessible storage medium (e.g., non-transitory memory) that when executed by a machine (e.g., a computer, a computing device, or otherwise), will cause the machine to perform operations for re-identification of an area of interest in an endoscopy procedure (e.g., a colonoscopy).

As illustrated in FIG. 3, method 300 includes blocks 301-327. However, it is appreciated that the order in which some or all of the process blocks appear in the method 300 should not be deemed limiting. Rather, one of ordinary skill in the art having the benefit of the present disclosure will understand that some of the process blocks may be executed in a variety of orders not illustrated, or even in parallel.

In an embodiment, method 300 begins with process block 301, which includes identifying a first area of interest. In an embodiment, identifying a first area of interest includes using a machine-learning model trained to identify one or more particular types of areas of interest. In an embodiment, the first area of interest is a polyp, such as may be identified during a colonoscopy. In an embodiment, identifying the first area of interest is in response to or based on an input from a user, such as an operator of an endoscopic system annotating one or more image frames of an endoscopy video feed. In an embodiment, the first area of interest comprises or is believed to comprise a polyp. In an embodiment, process block 301 is optional.

In an embodiment, process block 301 is followed by or method 300 begins with process block 303, which includes generating, with an image sensor, a first plurality of image frames of the first area of interest of the portion of the body. In an embodiment, generating, with the image sensor, the first plurality of image frames of the first area of interest of the portion of the body comprises generating a first video of the first area of interest, and wherein the first plurality of image frames are sequential image frames from the first video. In an embodiment, process block 303 includes receiving a first plurality of images of the first area of interest, rather than, for example, generating such images. In an embodiment, the first plurality of image frames is associated with a video of an endoscopy procedure (e.g., a colonoscopy procedure). The video may correspond to a video feed from an endoscope (e.g., a live video feed received during an endoscopy procedure) or a video obtained through other means (e.g., through a video database that includes endoscopy videos or any other computer or machine-readable medium containing endoscopy videos). It is appreciated that in some embodiments the resolution of the image frame may be equivalent to the original video (e.g., the image frame is a full-resolution image frame). In the same or other embodiments, the image frame may correspond to a down-sampled image frame (e.g., reduced resolution relative to the originally captured video) or up-sampled image frame (e.g., increased resolution relative to the originally captured video). The image frame may have a color (e.g., RGB, CMYK, or otherwise), grayscale, or black and white color space. It is appreciated that in some embodiments the color space of the image frame may match the color space from the video from which the image frame is obtained or be processed (e.g., extract grayscale images from a color video). In an embodiment, process block 303 is optional.

In an embodiment, process block 301 or 303 is followed by process block 305, which is shown to include tracking the first area of interest through the first plurality of image frames. As discussed further herein, the method 300 includes analyzing several image frames of an area of interest, such as from multiple angles, which is suitable to better characterize and/or re-identify the area of interest in other pluralities of image frames. Accordingly, in an embodiment, a first image frame of the first plurality of image frames is obtained at a first angle relative to the first area of interest, and a second image frame of the first plurality of image frames is obtained at a second angle relative to the first area of interest, wherein the first angle is different than the second angle. By tracking the area of interest through the first plurality of image frames, the method 300 is suitable identify which image frames include the first area of interest, thus improving characterization and re-identification of the area of interest. In an embodiment, process block 305 is optional.

In an embodiment, process blocks 301-305 is/are followed by process block 307, which includes transforming, with an embedding model, the first plurality of image frames to provide a first plurality of latent representations of low dimensionality based on the first plurality of image frames. Such transformation can include extracting different features from the image frame. The different features may be extracted to reduce the dimensionality of the image frame while simultaneously characterizing the image frame in a manner that can be compared with other image frames in the video or used to determine one or more attributes (e.g., whether the area of interest is in the image frame) with a reduced computational burden. It is appreciated that by extracting features from the image frame, a reduced dimensional representation (e.g., corresponding to the features) of the image frame may be utilized to characterize the first area of interest.

In some embodiments, the different features are extracted with a machine learning model or algorithm, such as including a plurality of deep neural networks. In one embodiment, the machine learning model will output the different features characterizing the image frame in response to the machine learning model receiving the image frame. In some embodiments, the different features are determined on a per-frame basis with each feature included in the different features associated with a corresponding neural network included in the plurality of deep neural networks.

In an embodiment, process block 307 is followed by process block 309, which is shown to include processing, with a multi-frame embedder, the first plurality of latent representations to provide a first embedding representation based on the first plurality of latent representations. In an embodiment, processing the first plurality of latent representations includes using an attention model to provide a dynamic interpretation of distinctive features of the first area of interest and the second area of interest. The multi-frame embedder receives as input the embedding vectors of multiple image frames, and mutually processes them to provide a single embedding representing the first area of interest as a combination of all provided frames. In this regard, the output of the multi-frame embedder is a single vector for the first area of interest.

Process blocks 311-319 are process blocks corresponding, respectively, to process blocks 301-309 and relate to a second area of interest in the portion of the body. In this regard, whereas process block 301 relates to identifying a first area of interest, process block 311 relates to identifying a second area of interest in the portion of the body. Similarly, in an embodiment, process block 313 relates to a process of generating a second plurality of image frames of the second area of interest analogous to process block 303. Likewise, in an embodiment, process block 315 includes tracking the second area of interest in the second plurality of image frames analogous to the tracking process described further herein with respect to process block 305. Moreover, in an embodiment, process block 317 includes transforming, with the embedding model, the second plurality of images frames to provide a second plurality of latent representations of low dimensionality based on the second plurality of image frames, which is similar or analogous to the process of transforming described herein with respect to process block 307. Lastly, in an embodiment, process block 319 includes processing, with the multi-frame embedder, the second plurality of latent representations to provide a second embedding representation based on the second plurality of latent representations, which can include analogous processes as those described with respect to process block 309.

In an embodiment, process blocks 311-319 relating to identifying a second area of interest (311), generating a second plurality of image frames (313), tracking the second area of interest (315), transforming the second plurality of image frame (317), processing a second plurality of latent representations (319), and processing the second plurality of latent representations to provide a second embedding representation (321), occur during a different phase of a procedure (i.e., insertion vs. removal) than process blocks 301-309, such as during a different and/or separate period of time. As an example, in an embodiment, generating the first plurality of image frames occurs before generating the second plurality of image frames, such as where the first plurality of image frames is obtained while the endoscope is being inserted into the portion of the body, and the second plurality of image frames are obtained while the endoscope is being removed from the portion of the body. In an embodiment, the second area of interest is of a same class or category as the first area of interest, such as where both the first area of interest and the second area of interest are each polyps observed during a colonoscopy.

Process block 319 is followed by process block 321, which includes comparing, with a classifier, the first embedding representation and the second embedding representation. In an embodiment, comparing, with the classifier, the first embedding representation and the second embedding representation comprises generating a cosine similarity score of the first embedding representation and the second embedding representation. In this regard, in an embodiment, the classifier takes as input two vectors of multi-frame representations and based on the cosine similarity score determines whether they belong to the same area of interest or not.

In an embodiment, process block 321 is followed by process block 323, which includes determining a likelihood that the first area of interest is the second area of interest based on the comparison between the first embedding representation and the second embedding representation. While, in an embodiment, such a determination is part of process block 319, in certain embodiments, the process of determining is part of a separate process block. In an embodiment, process block 323 is optional.

In an embodiment, process block 323 includes displaying or otherwise indicating a probability score that the second area of interest is the same area of interest (i.e., the first area of interest) that was previously identified. In an embodiment, process block 323 includes indicating that the area of interest is the same area of interest that was previously identified.

In an embodiment, the method 300 includes steps to improve the predictive capabilities of the method, such as through process blocks 325 and 327. As discussed further herein with respect to process blocks 301 and 305, in an embodiment, the method 300 includes identifying an area of interest; and tracking the area of interest through a plurality of image frames. As the area of interest is identified and tracked through a series of image frames, it is known that the area of interest through the series of image frames is the same area of interest throughout the series of image frames.

Accordingly, a comparison of one subset of the image frames and another subset of the image frames can be used to inform and improve the classifier, discussed further herein with respect to process block 321. In this regard, in an embodiment, process block 325 includes comparing a first subset of the plurality of image frames with a second subset of the plurality of image frames. In an embodiment, the first subset can be further compared, such as with the classifier, with a plurality of images of portions of a body known not to be of the area of interest, such as from another body, another area of interest, etc. Then, the classifier is updated based (as in process block 327) on the comparison between the first and second subset, and, in certain embodiments, the comparison with plurality of images of portions of a body known not to be of the area of interest.

In an embodiment, the first subset of the plurality of images is from a first period of time and the second subset of the plurality of images is from a second period of time different, distinct, and not overlapping with the first period of time. By comparing images of the same area of interest from different and non-overlapping times, the classifier can be better improved than if the periods of time were overlapping, as the first and second subsets of image frames do not include common image frames.

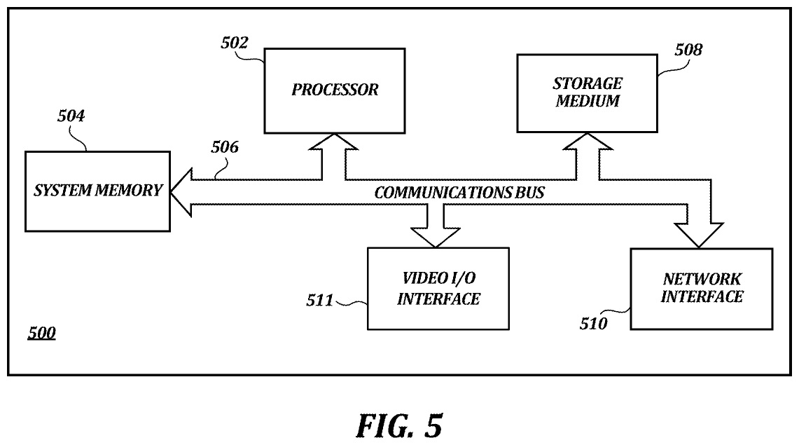

FIG. 5 is a block diagram that illustrates aspects of a demonstrative computing device 500 appropriate for implementing EVA 115 illustrated in FIG. 1B, the method illustrated in FIG. 2, or the method 300 illustrated in FIG. 3, in accordance with embodiments of the present disclosure. Those of ordinary skill in the art will recognize that computing device 500 may be implemented using currently available computing devices or yet to be developed devices.

In its most basic configuration, computing device 500 includes at least one processor 502 and a system memory 504 connected by a communication bus 506. Depending on the exact configuration and type of device, system memory 504 may be volatile or nonvolatile memory, such as read only memory (“ROM”), random access memory (“RAM”), EEPROM, flash memory, or similar memory technology. Those of ordinary skill in the art will recognize that system memory 504 typically stores data and/or program modules that are immediately accessible to and/or currently being operated on by the processor 502. In this regard, the processor 502 may serve as a computational center of computing device 500 by supporting the execution of instructions.

As further illustrated in FIG. 5, computing device 500 may include a network interface 510 comprising one or more components for communicating with other devices over a network. Embodiments of the present disclosure may access basic services that utilize network interface 510 to perform communications using common network protocols. Network interface 510 may also include a wireless network interface configured to communicate via one or more wireless communication protocols, such as WiFi, 2G, 3G, 4G, LTE, WiMAX, Bluetooth, and/or the like.

In the exemplary embodiment depicted in FIG. 5, computing device 500 also includes a storage medium 508. However, services may be accessed using a computing device that does not include means for persisting data to a local storage medium. Therefore, the storage medium 508 may be omitted. In any event, the storage medium 508 may be volatile or nonvolatile, removable or nonremovable, implemented using any technology capable of storing information such as, but not limited to, a hard drive, solid state drive, CD-ROM, DVD, or other disk storage, magnetic cassettes, magnetic tape, magnetic disk storage, and/or the like.

The illustrated embodiment of computing device 500 further includes a video input/out interface 511. Video I/O interface 511 may include an analog video input (e.g., composite video, component video, VGG connector, etc.) or a digital video input (e.g., HDMI, DVI, DisplayPort, USB-A, USB-C, etc.) to receive the live video feed from colonoscope 105 and a similar type of video output port to output the live video feed within colonoscopy UI to display 110. In one embodiment, video I/O interface 511 may also represent a graphics processing unit capable of performing the necessary computational video processing to generate and render colonoscopy UI.

As used herein, the term “computer-readable medium” includes volatile and non-volatile and removable and non-removable media implemented in any method or technology capable of storing information, such as computer-readable instructions, data structures, program modules, or other data.

Suitable implementations of computing devices that include a processor 502, system memory 504, communication bus 506, storage medium 508, and network interface 510 are known and commercially available. For ease of illustration and because it is not important for an understanding of the claimed subject matter, FIG. 5 does not show some of the typical components of many computing devices. In this regard, the computing device 500 may include input devices, such as a keyboard, keypad, mouse, microphone, touch input device, touch screen, tablet, and/or the like. Such input devices may be coupled to computing device 500 by wired or wireless connections including RF, infrared, serial, parallel, Bluetooth, USB, or other suitable connection protocols using wireless or physical connections. Since these devices are well known in the art, they are not illustrated or described further herein.

The above user-interface has been described in terms of a colonoscopy and is particularly well-suited as a colonoscopy user-interface to aid visualization of colonoscopy procedures and/or analysis of colonoscopy videos. However, it should be appreciated that user-interface may be more broadly/generically described as an endoscopy user-interface that may be used to visualize endoscopy procedures, in general, related to other anatomical structures and/or analyze endoscopy videos. For example, the method 300 is applicable for analyzing other gastroenterological procedures including endoscopy procedures within the upper and lower gastrointestinal tracts. In yet other examples, the method 300 may be used to analyze videos of non-gastroenterological procedures that may occur in the esophagus, bronchial tubes, other tube-like anatomical structures, etc.

The processes and user-interface described above are described in terms of computer software and hardware. The techniques described may constitute machine-executable instructions embodied within a tangible or non-transitory machine (e.g., computer) readable storage medium, that when executed by a machine will cause the machine to perform the operations described. Additionally, some of the processes or logic for implementing the user-interface may be embodied within hardware, such as an application specific integrated circuit (“ASIC”) or otherwise.

A tangible machine-readable storage medium includes any mechanism that provides (i.e., stores) information in a non-transitory form accessible by a machine (e.g., a computer, network device, personal digital assistant, manufacturing tool, any device with a set of one or more processors, etc.). For example, a machine-readable storage medium includes recordable/non-recordable media (e.g., read only memory (ROM), random access memory (RAM), magnetic disk storage media, optical storage media, flash memory devices, etc.).

The above description of illustrated embodiments of the invention, including what is described in the Abstract, is not intended to be exhaustive or to limit the invention to the precise forms disclosed. While specific embodiments of, and examples for, the invention are described herein for illustrative purposes, various modifications are possible within the scope of the invention, as those skilled in the relevant art will recognize.

These modifications can be made to the invention in light of the above detailed description. The terms used in the following claims should not be construed to limit the invention to the specific embodiments disclosed in the specification. Rather, the scope of the invention is to be determined entirely by the following claims, which are to be construed in accordance with established doctrines of claim interpretation.

The embodiments of the invention in which an exclusive property or privilege is claimed are defined as follows:

1. An endoscopic system comprising:
an endoscope configured to enter a portion of a body, the endoscope comprising an image sensor configured to image the portion of the body; and
a controller operatively coupled to the image sensor, the controller including logic that, when executed, causes the endoscopic system to perform operations comprising:
transforming, with an embedding model, a first plurality of image frames of a first area of interest of the portion of the body to provide a first plurality of latent representations of low dimensionality based on the first plurality of image frames;
transforming, with the embedding model, a second plurality of images frames of a second area of interest of the portion of the body to provide a second plurality of latent representations of low dimensionality based on the second plurality of image frames;
processing, with a multi-frame embedder, the first plurality of latent representations to provide a first embedding representation based on the first plurality of latent representations;
processing, with the multi-frame embedder, the second plurality of latent representations to provide a second embedding representation based on the second plurality of latent representations; and
comparing, with a classifier, the first embedding representation and the second embedding representation.

2. The endoscopic system of claim 1, wherein the controller comprises logic that, when executed, causes the endoscopic system to perform operations comprising:
determining a likelihood that the first area of interest is the second area of interest based on the comparison between the first embedding representation and the second embedding representation.

3. The endoscopic system of claim 1, wherein comparing, with the classifier, the first embedding representation and the second embedding representation comprises generating a cosine similarity score of the first embedding representation and the second embedding representation.

4. The endoscopic system of claim 1, wherein the controller comprises logic that, when executed, causes the endoscopic system to perform operations comprising:
comparing a first subset of the first plurality of image frames with a second subset of the image frames; and
updating the classifier based on comparing the first subset and the second subset.

5. The endoscopic system of claim 1, wherein processing the first plurality of latent representations and the second plurality of representations with the multi-frame embedder comprises using an attention model to provide a dynamic interpretation of distinctive features of the first area of interest and the second area of interest.

6. The endoscopic system of claim 1, wherein the controller comprises logic that, when executed, causes the endoscopic system to perform operations comprising:
identifying the first area of interest;
tracking the first area of interest through the first plurality of image frames;
identifying the second area of interest; and
tracking the second area of interest through the second plurality of image frames.

7. The endoscopic system of claim 1, wherein the controller comprises logic that, when executed, causes the endoscopic system to perform operations comprising:
generating, with the image sensor, the first plurality of image frames of the first area of interest of the portion of the body;
generating, with the image sensor, the second plurality of image frames of the second area of interest of the portion of the body;
wherein generating, with the image sensor, the first plurality of image frames of the first area of interest of the portion of the body comprises generating a first video of the first area of interest, and wherein the first plurality of image frames are sequential image frames from the first video, and
wherein generating, with the image sensor, the second plurality of image frames of the second area of interest of the portion of the body comprises generating a second video of the second area of interest, and wherein the second plurality of image frames are sequential image frames from the second video.

8. A computer-implemented method of analyzing a portion of a body, the method comprising:
transforming, with an embedding model, a first plurality of image frames of a first area of interest of a portion of the body to provide a first plurality of latent representations of low dimensionality based on the first plurality of image frames;
transforming, with the embedding model, a second plurality of image frames of a second area of interest of the portion of the body to provide a second plurality of latent representations of low dimensionality based on the second plurality of image frames;
processing, with a multi-frame embedder, the first plurality of latent representations to provide a first embedding representation based on the first plurality of latent representations;
processing, with the multi-frame embedder, the second plurality of latent representations to provide a second embedding representation based on the second plurality of latent representations; and
comparing, with a classifier, the first embedding representation and the second embedding representation.

9. The computer-implemented method of claim 8, further comprising determining a likelihood that the first area of interest is the second area of interest based on the comparison between the first embedding representation and the second embedding representation.

10. The computer-implemented method of claim 8, wherein the controller comprises logic that, when executed, causes the endoscopic system to perform operations comprising:
comparing a first subset of the first plurality of image frames with a second subset of the image frames; and
updating the classifier based on comparing the first subset and the second subset.

11. The computer-implemented method of claim 8, wherein comparing, with the classifier, the first embedding representation and the second embedding representation comprises generating a cosine similarity score using the first embedding representation and the second embedding representation as inputs.

12. The computer-implemented method of claim 8, wherein processing the first plurality of latent representations and the second plurality of representations comprises using an attention model to provide a dynamic interpretation of distinctive features of the first area of interest and the second area of interest.

13. The computer-implemented method of claim 8, the method further comprises:
identifying the first area of interest;
tracking the first area of interest through the first plurality of image frames;
identifying the second area of interest; and
tracking the second area of interest through the second plurality of image frames.

14. The computer-implemented method of claim 8, wherein the first area of interest comprises or is believed to comprise a polyp, and wherein the second area of interest comprises or is believed to comprise a polyp.

15. The computer-implemented method of claim 8, wherein a first image frame of the first plurality of image frames is obtained at a first angle relative to the first area of interest, wherein a second image frame of the first plurality of image frames is obtained at a second angle relative to the first area of interest, and wherein the first angle is different than the second angle.

16. The computer-implemented method of claim 8, further comprising:
generating, with an image sensor positioned at a distal end of an endoscope, the first plurality of image frames of a first area of interest of a portion of the body;
generating, with the image sensor, a second plurality of image frames of the second area of interest of the portion of the body;
wherein generating the first plurality of image frames occurs before generating the second plurality of image frames.

17. The computer-implemented method of claim 8, wherein the first plurality of image frames is obtained while the endoscope is being inserted into the portion of the body, and wherein the second plurality of image frames are obtained while the endoscope is being removed from the portion of the body.

18. At least one machine-accessible storage medium that provides instructions that, when executed by a machine, will cause the machine to perform operations comprising:
transforming, with an embedding model, a first plurality of image frames of a first area of interest of a portion of a body to provide a first plurality of latent representations of low dimensionality based on the first plurality of image frames;
transforming, with the embedding model, a second plurality of image frames of a second area of interest of the portion of the body to provide a second plurality of latent representations of low dimensionality based on the second plurality of image frames;
processing, with a multi-frame embedder, the first plurality of latent representations to provide a first embedding representation based on the first plurality of latent representations;
processing, with the multi-frame embedder, the second plurality of latent representations to provide a second embedding representation based on the second plurality of latent representations; and
comparing, with a classifier, the first embedding representation and the second embedding representation.

19. The at least one machine-accessible storage medium of claim 18, that provides further instructions that, when executed by a machine, will cause the machine to perform operations comprising:
determining a likelihood that the first area of interest is the second area of interest based on the comparison between the first embedding representation and the second embedding representation.

20. The at least one machine-accessible storage medium of claim 18, wherein comparing, with the classifier, the first embedding representation and the second embedding representation comprises generating a cosine similarity score of the first embedding representation and the second embedding representation.
